(19) United States
(12) Patent Application Publication
(10) Pub. No.: US 2006/0039956 A1
(43) Pub. Date: Feb. 23, 2006

(54) Impregnating solution for cosmetic cloths

(76) Inventors: Hermann Hensen, Haan (DE); Virginia Lazarowitz, Hatfield, PA (US); Timothy Morris, Morton, PA (US); Heike Kublik, Kempen (DE); Jackie Searle, Kent (GB); Mark Leonard, Kent (GB)

Correspondence Address: COGNIS CORPORATION;PATENT DEPARTMENT, 300 BROOKSIDE AVENUE, AMBLER, PA, 19002, US

(21) Appl. No.: 10/493,554

(22) PCT Filed: Oct. 18, 2002
(86) PCT No.: PCT/EP02/11674

§ 371 Date: Jan. 6, 2005

(30) Foreign Application Priority Data
Oct. 26, 2001 (DE) 101 52 942.2
Dec. 18, 2001 (DE) 101 62 184.1

(51) Int. Cl. A61K 9/70; A61K 8/73

(52) U.S. Cl. 424/443; 424/70.13

(57) ABSTRACT

The invention relates to preparations for impregnating cosmetic wipes, characterized in that they contain (a) an emulsifier mixture containing nonionic and amphoteric surfactants in a quantity ratio of 10:1 to 1:1, based on the quantity of emulsifiers, (b) a mixture of wax components containing wax esters, partial glycerides and fatty alcohol ethoxylates and (c) at least one cationic polymer. The preparations containing wax particles lead to optimal foaming and cleansing performance and to a favorable sensorial impression.

### FIELD OF THE INVENTION

This invention relates generally to cosmetics and more especially to impregnating solutions containing certain skin care and cleansing agents with particulate waxes for application to cosmetic wipes with a particular structure.

### PRIOR ART

Cosmetic wipes are becoming increasingly important as a simple and hygienic way of meeting the needs of modern personal care. Two forms of cosmetic wipes are already being marketed by different manufacturers: moist wipes made of woven fabric or even tissue paper which are impregnated with a cleansing or skin-care formulation and dry wipes which have to be moistened before use. Numerous patent applications describe cleansing cloths where solutions are absorbed onto various fabrics. Starting out from the cleansing function demanded of the first marketable cosmetic wipes, skin care is now increasingly the focus of attention. For example, International patent application WO 95/35411 proposes moist wipes impregnated with a lotion which, besides mineral oil, contains fatty acid esters, fatty alcohol ethoxylates and fatty alcohols. International patent applications WO 99/13861 and WO 01/08657 are cited as representative of dry cosmetic wipes. In their case, nonwoven generally structureless fabrics are impregnated and/or coated with formulations containing surfactants and skin care additives and are then optionally dried. Depending on the size of the carrier, the wipes in question can be impregnated non-reusable wash cloths or even relatively small cleansing pads. Consumer demands as to cleansing performance, skin feel during and after use and the easy-to-use aspect represent a technical challenge to the developer. Thus, the foam volume generated after wetting or in the moistened wipes themselves, foam stability and foam structure have a critical bearing on the sensorial impression during and after cleansing. A suitable foam structure and an adequate foam volume after wetting are difficult to achieve without subjecting the cloth/wipe to vigorous mechanical rubbing between the hands. This problem can be exacerbated by other non-surfactant skin care additives.

Accordingly, the problem addressed by the present invention was to provide formulations for the impregnation of moist and dry cosmetic wipes with improved cleansing, skin care and sensorial properties for the cleansing and care of the hair and body. In particular, the formulations according to the invention would have a deep cleansing effect on the skin pores and would achieve a pleasant skin feel during and after use supported by the optimized foaming properties of the formulation.

### DESCRIPTION OF THE INVENTION

The present invention relates to preparations for impregnating cosmetic wipes which are characterized in that they contain
(a) an emulsifier mixture containing nonionic and amphoteric surfactants in a quantity ratio of 10:1 to 1:1, based on the quantity of emulsifiers,
(b) a mixture of wax components containing wax esters, partial glycerides and fatty alcohol ethoxylates and
(c) at least one cationic polymer.

It has been found that formulations with the composition shown above on the structured surface of a carrier (cosmetic wipe) lead to advantageous foaming, a good cleansing effect and a pleasant skin feel. Through the combination of the carrier with impregnating solutions containing “caring”, finely dispersed, particulate wax dispersions of a certain particle size, a fine-cell voluminous foam can be rapidly generated by gentle mechanical action. The composition of wax components containing wax esters, partial glycerides and fatty alcohol ethoxylates has proved particularly successful in this regard, as has an emulsifier mixture of nonionic and amphoteric surfactants in a quantity ratio of 10:1 to 1:1. Impregnating solutions with this composition showed a particularly good skin feel during and after the use of the correspondingly impregnated cosmetic wipes. In addition, the size of the wax particles has a bearing on the properties of the foam and the skin feel during and after cleansing. The smaller the particles are in size, the more pleasant the sensorial impression.

Carriers

The impregnating solutions according to the invention are suitable for application to moist cosmetic wipes although they are preferably used on dry cosmetic wipes.

The special carrier systems to which the present invention relates may have a single-ply or multi-ply structure. Besides paper-based tissues, corresponding tissue cloths made of fibers or fleeces are also suitable. Examples of natural fibers include silk, cellulose, keratin, wool, cotton, jute, linen, flax; examples of synthetic fibers include acetate, acrylate, cellulose ester, polyamide, polyester, polyolefin, polyvinyl alcohol, polyurethane fibers or even additive-hydrophilicized polyolefin fabrics and blends of these fibers or fabrics. Reaction products of 1 part polyethylene glycol with 2 parts C₁₀₋₁₂ fatty acids or derivatives thereof are used to hydrophilicize the polyolefin-containing fabrics.

Nonwoven fabrics are preferred because they can be better provided with the structure required in accordance with the invention. Carriers of viscose/polyester blends are particularly suitable. However, hydroentangled carrier systems of 50 to 90% by weight viscose and 50 to 10% by weight polyester are preferred, carriers of 60 to 80% by weight viscose and 40 to 20% by weight polyester being particularly preferred and those of 65 to 70% by weight viscose and 35 to 30% by weight polyester being most particularly preferred.

In terms of size, the wipes are generally between 100 and 500 mm in length and between 100 and 500 mm in width, lengths and widths of 120 to 220 mm being preferred. However, the fabric may even be in the form of a glove and, in that case, may possibly have a multi-ply structure so that the inner fabric layer of the glove is more hydrophobic, has a barrier function and protects the hand against contact with the formulation or with moisture.

By virtue of their production (hydroentanglement with a hydroentanglement belt), the carrier fabrics of the cosmetic wipes according to the invention have a uniformly structured surface with round to oval depressions. These depressions—also known as pits—are round to oval in shape with a diameter or width of 0.1 to 1 mm and preferably 0.2 to 0.6 mm and a diameter or length of 0.5 to 5.0 mm and preferably 0.8 to 1.5 mm. They may be present on both sides or on only one side. Where they are present on one side, the depressions occupy between 50 and 99% and preferably between 60 and 85% of the thickness of the carrier. Where the depressions are present on both sides, this percentage has to be divided up accordingly. On average, between 500 and 4,000, preferably between 1,500 and 3,500 and more preferably between 2,500 and 3,200 pits are present per 100 mm² carrier surface area.

Impregnating Solutions

Impregnating solutions—also known as coating solutions—are understood to be the preparations according to the invention in the form of solutions, dispersions and emulsions which are applied to the carriers for cosmetic wipes.

The ratio by weight of dry cloth to applied cleansing and skin care solution is intended to be 60:0 to 90:10 and preferably 85:15 to 80:20. The impregnating solution contains suractants and wax dispersions with mean particle sizes of of up to at most 13 μm, preferably at most 4 μm and more preferably at most 2 μm.

Emulsifier Mixtures

The emulsifier mixture of which the total content in the impregnating or coating solutions is normally about 1.5 to 75, preferably 15 to 55 and more preferably 25 to 40% by weight contains nonionic, amphoteric and optionally anionic surfactants. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow homolog distribution. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Information on their structure and production can be found in relevant synoptic works, cf. for example J. Falbe (ed.), “Surfactants in Consumer Products”, Springer Verlag, Berlin, 1987, pages 54 to 124 or J. Falbe (ed.), “Katalysatoren, Tenside und Mineral-öladditive (Catalysts, Surfactants and Mineral Oil Additives)”, Thieme Verlag, Stuttgart, 1978, pages 123-217.

Typical examples of particularly suitable mild, i.e. particularly dermatologically safe, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acid, alkyl oligoglucosides, fatty acid glucamides, alkyl amidobetaines, amphoacetates and/or protein fatty acid condensates—the latter preferably based on wheat proteins.

Preferred surfactants for the purposes of the present invention are Disodium Cocoamphodiacetate, Sodium Cocoamphoacetate, Cocamidopropyl Betaine, Cocamide DEA, alkyl oligoglucosides and mixtures thereof.

Particularly preferred surfactants are nonionic surfactants selected from the group consisting of alkyl oligoglucosides, Cocamidopropyl Betaine, PEG-7, Glyceryl Cocoate, Laureth-4, Ceteareth-12, Ceteareth-20 and/or Beheneth-10, surfactant mixtures of alkyl oligoglycosides and betaines, especially Cocamidopropyl Betaine, in a quantity ratio of 10:1 to 1:1, preferably 5:1 to 1.5:1 and more preferably 4:1 to 2:1 being most particularly preferred.

Alkyl and alkenyl oligoglycosides are known nonionic surfactants which correspond to formula (I):
R¹O-[G]ₚ (I)
where R¹ is an alkyl and/or alkenyl group containing 4 to 22 carbon atoms, G is a sugar unit containing 5 or 6 carbon atoms and p is a number of 1 to 10. They may be obtained by the relevant methods of preparative organic chemistry. The overviews presented by Bierman et al. in Starch/Stärke 45, 281 (1993), by B. Salka in Cosm. Toil. 108, 89 (1993) and by J. Kahre in SÖFW-Journal No. 8, 598 (1995) are cited as representative of the extensive literature available on this subject.

The alkyl and/or alkenyl oligoglycosides may be derived from aldoses or ketoses containing 5 or 6 carbon atoms, preferably glucose. Accordingly, the preferred alkyl and/or alkenyl oligoglycosides are alkyl and/or alkenyl oligoglucosides. The index p in general formula (I) indicates the degree of oligomerization (DP), i.e. the distribution of mono- and oligoglycosides, and is a number of 1 to 10. Whereas p in a given compound must always be an integer and, above all, may assume a value of 1 to 6, the value p for a certain alkyl oligoglycoside is an analytically determined calculated quantity which is generally a broken number. Alkyl and/or alkenyl oligoglycosides having an average degree of oligomerization p of 1.1 to 3.0 are preferably used. Alkyl and/or alkenyl oligoglycosides having a degree of oligomerization of less than 1.7 and, more particularly, between 1.2 and 1.4 are preferred from the applicational point of view. The alkyl or alkenyl radical R¹ may be derived from primary alcohols containing 4 to 11 and preferably 8 to 10 carbon atoms. Typical examples are butanol, caproic alcohol, caprylic alcohol, capric alcohol and undecyl alcohol and the technical mixtures thereof obtained, for example, in the hydrogenation of technical fatty acid methyl esters or in the hydrogenation of aldehydes from Roelen's oxosynthesis. Alkyl oligoglucosides having a chain length of C₈ to C₁₀ (DP=1 to 3), which are obtained as first runnings in the separation of technical C₈₋₁₈ coconut oil fatty alcohol by distillation and which may contain less than 6% by weight of C₁₂ alcohol as an impurity, and also alkyl oligoglucosides based on technical C_{9/11} oxoalcohols (DP=1 to 3) are preferred. In addition, the alkyl or alkenyl radical R¹ may also be derived from primary alcohols containing 12 to 22 and preferably 12 to 14 carbon atoms. Typical examples are lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and technical mixtures thereof which may be obtained as described above. Alkyl oligoglucosides based on hydrogenated C_{12/14} coconut oil fatty alcohol having a DP of 1 to 3 are preferred.

Betaines are known surfactants which are mainly produced by carboxyalkylation, preferably carboxymethylation, of aminic compounds. The starting materials are preferably condensed with halocarboxylic acids or salts thereof, more particularly with sodium chloroacetate, 1 mole of salt being formed per mole of betaine. The addition of unsaturated carboxylic acids, for example acrylic acid, is also possible. Particulars of the nomenclature and, in particular, the distinction between betaines and Agenuine@ amphoteric surfactants can be found in the article by U. Ploog in Seifen-Öle-Fette-Wachse, 198, 373 (1982). Other reviews of this subject have been published, for example, by A. O'Lenick et al. in HAPPI, Nov. 70 (1986), by S. Holzman et al. in Tens. Surf. Det. 23, 309 (1986), by R. Bibo et al. in Soap Cosm. Chem. Spec., Apr. 46 (1990) and by P. Ellis et al. in Euro Cosm. 1, 14 (1994). Examples of suitable betaines are the carboxyalkylation products of secondary and, in particular, tertiary amines corresponding to formula (II): 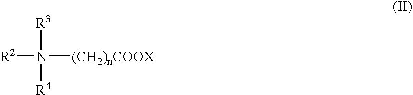
in which R² stands for alkyl and/or alkenyl groups containing 6 to 22 carbon atoms, R³ stands for hydrogen or alkyl groups containing 1 to 4 carbon atoms, R⁴ stands for alkyl groups containing 1 to 4 carbon atoms, n is a number of 1 to 6 and X is an alkali metal and/or alkaline earth metal or ammonium. Typical examples are the carboxymethylation products of hexyl methyl amine, hexyl dimethyl amine, octyl dimethyl amine, decyl dimethyl amine, dodecyl methyl amine, dodecyl dimethyl amine, dodecyl ethyl methyl amine, C_{12/14} cocoalkyl dimethyl amine, myristyl dimethyl amine, cetyl dimethyl amine, stearyl dimethyl amine, stearyl ethyl methyl amine, oleyl dimethyl amine, C_{16/18} tallow alkyl dimethyl amine and technical mixtures thereof.

Other suitable betaines are carboxyalkylation products of amidoamines corresponding to formula (III): 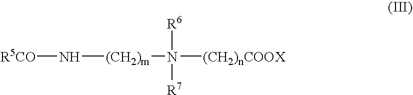
in which R⁵CO is an aliphatic acyl group containing 6 to 22 carbon atoms and 0 or 1 to 3 double bonds, m is a number of 1 to 3 and R⁶, R⁷, n and X are as defined above. Typical examples are reaction products of fatty acids containing 6 to 22 carbon atoms, namely caproic acid, caprylic acid, capric acid, lauric acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof, with N,N-dimethyl aminoethyl amine, N,N-dimethyl aminopropyl amine, N,N-diethyl aminoethyl amine and N,N-diethyl aminopropyl amine which are condensed with sodium chloroacetate. It is preferred to use a condensation product of C_{8/18} cocofatty acid-N,N-dimethyl aminopropyl amide with sodium chloroacetate.

Besides the nonionic and amphoteric surfactants compulsorily present, the impregnating solutions according to the invention contain other surfactants, such as anionic or cationic surfactants. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and di-alkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of cationic surfactants are quaternary ammonium compounds and esterquats, more particularly quaternized fatty acid trialkanolamine ester salts.

Of the other surfactants optionally used, the anionic surfactants (component e)) are preferred. They are used in a quantity ratio to the amphoteric surfactants of 0 to 1:1 and preferably 0.3 to 0.6:1, based on the amphoteric surfactants.

Wax Dispersions

The percentage content of the wax components present in the preparations in the impregnating and coating solutions according to the invention is 0.2 to 35% by weight, preferably 1 to 25% by weight and more preferably 2 to 20% by weight.

The wax mixtures according to the invention contain wax esters, partial glycerides and fatty alcohol ethoxylates. Other wax components which may be present include substances from the groups of alkylene glycol esters, fatty acid alkanolamides, triglycerides, esters of polybasic and/or monobasic, optionally hydroxysubstituted carboxylic acids, fatty alcohols, fatty alcohols, fatty ketones, fatty acids, fatty aldehydes, fatty ethers, fatty carbonates, ring opening products of olefin epoxides and mixtures thereof.

The wax esters are normally esters of monobasic and polybasic, branched and unbranched, saturated and unsaturated, optionally hydroxysubstituted carboxylic acids with fatty alcohols containing 6 to 22 carbon atoms. The acid component of these esters may be selected, for example, from palmitic acid, palmitoleic acid, stearic acid, isostearic acid, oleic acid, malonic acid, maleic acid, fumaric acid, adipic acid, sebacic acid, azelaic acid, dodecanedioic acid, phthalic acid, isophthalic acid, succinic acid and also malic acid, citric acid and, more particularly, tartaric acid and mixtures thereof. The fatty alcohols contain 6 to 22, preferably 12 to 18 and more preferably 16 to 18 carbon atoms in the alkyl chain. Typical examples are caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and technical mixtures thereof. The esters may be present as full or partial esters. Esters of linear saturated fatty acids with cetyl alcohol, especially cetyl palmitate, are preferably used.

Partial glycerides are monoesters and/or diesters of glycerol with linear and branched, saturated and unsaturated fatty acids, i.e. for example caproic acid, caprylic acid, capric acid, lauric acid, myristic acid, palmitic acid, palmitoleic acid, stearic acid, isostearic acid, behenic acid and technical mixtures thereof. They correspond to formula (IV): 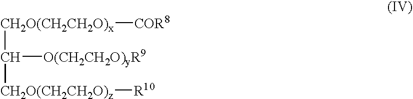
in which R⁸CO is a linear and b ranched, saturated and unsaturated acyl group containing 6 to 22 carbon atoms, R⁹ and R¹⁰ independently of one another represent hydrogen or have the same meaning as R⁸CO, x, y and z together stand for 0 or for a number of 1 to 30. Typical examples are lauric acid monoglyceride, oleic acid monoglyceride, lauric acid diglyceride, coconut fatty acid monoglyceride, coconut fatty acid triglyceride, palmitic acid monoglyceride, palmitic acid triglyceride, stearic acid monoglyceride, stearic acid diglyceride, tallow fatty acid monoglyceride, tallow fatty acid diglyceride, castor oil and hydrogenated castor oil, behenic acid monoglyceride, behenic acid diglyceride and technical mixtures thereof which may still contain small quantities of triglyceride from the production process.

A third group of suitable wax components in the wax mixtures are fatty alcohol ethoxylates corresponding to formula (V):
R¹¹O(CH₂—CH₂—O)—H (V)
in which R¹¹ is a linear, optionally hydroxysubstituted alkyl group containing 16 to 48 and preferably 18 to 36 carbon atoms. Typical examples of suitable alcohols are cetearyl alcohol, hydroxystearyl alcohol, behenyl alcohol and oxidation products of long-chain paraffin. Ethoxylated behenyl alcohols are particularly preferred wax components.
Cationic Polymers

Preferred other auxiliaries in the impregnating solutions are cationic polymers which are used in the impregnating solutions for the cosmetic wipes according to the invention in quantities of 0.02 to 3% by weight, preferably 0.05 to 1% by weight and more preferably in quantities of 0.1 to 1% by weight. Suitable cationic polymers include, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lame-quat® L, Grünau), quaternized wheat polypeptides, polyethyleneimine, cationic silicone polymers such as, for example, Amodimethicone, copolymers of adipic acid and dimethylaminohydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2252840 A and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol®A-15, Mirapol® AD-1, Mirapol® AZ-1 of Miranol.

Cationic guar gum and/or quaternized ammonium salt polymers are preferably used in the preparations according to the invention.

Polyols

Polyols which are optional component in the context of the invention preferably contain 2 to 15 carbon atoms and at least two hydroxyl groups. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 dalton;
technical oligoglycerol mixtures with a degree of self-condensation of 1.5 to 10 such as, for example, technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine.

Preferred polyols are glycerol and propylene glycol. They are used in quantities of 0 to 20% by weight, preferably 1 to 15% by weight and more particularly 3 to 12% by weight, based on the impregnating solutions.

Particle Sizes of the Wax Dispersions and Particle Size Determination

The particle sizes of the wax dispersions used in the impregnating and coating solutions have a major bearing on the sensorial properties and foaming and cleansing properties of the wipes. For optimum performance, the dispersions should have mean particle sizes of at most 13 μm, preferably at most 4 μm and more preferably at most 2 μm. The usual processes known to the expert for producing dispersions are selected for this purpose. Thus, the particle sizes may be adjusted, for example, by subjecting the dispersions to varying loads in high-pressure homogenizers.

However, the particular conditions for adjusting the mean particle sizes have to be adapted to the wax dispersions according to the wax components selected and any other auxiliaries. Since the reduction in particle size is generally accompanied by an increase in the temperature of the medium and since further processing of the wax dispersions is supposed to be possible without any change in particle size, the wax components selected should have a melting point or melting range of 55 to 90° C.

The particle sizes were determined with a Coulter LS 230 laser particle analyzer from Beckman Coulter. Besides conventional laser diffraction, this instrument also uses the PIDS (polarized intensity differential scattering) method for extending the measuring range to the submicron range. The laser produces light with a wavelength of 750 nm which is augmented by the wavelengths 450, 600 and 900 nm by the PIDS method. The 132 detectors combine to give an overall measuring range of 0.04 to 2,000 μm which is divided into 116 size classes. Evaluation is carried out by the software either on the basis of Frauenhofer's diffraction theory or on the basis of Mie's scattered light theory.

The measured samples are diluted by a factor of 100 by the sample feed module, deionized water being used as the dispersion medium. The sample feed module guarantees continuous circulation of the dispersed sample through the measuring cell. Before the beginning of the actual measurement, the circulating sample was ultrasonicated for 30 s. This was done by a Beckman Coulter ultrasound module connected to the analyzer. Evaluation was carried out exclusively in accordance with Mie's scattered light theory using a refractive index of 1.47 for the dispersed medium. The mean particle sizes mentioned are based on the D₅₀ value of the volume distribution.

Production Process

The impregnating solutions containing caring, finely dispersed, particulate wax dispersions with a certain particle size are applied to the structured fabric in such a way that, after the optional subsequent drying process by hot air drying, vacuum drying or roller drying, the dispersion adheres loosely to the carrier as a discontinuous layer. The temperature during the drying process has to be selected so that the temperature of the fabric stays below the melting point of the wax components in order to prevent the particles from fusing with the fabric and to enable the particles to be finely dispersed in the foam generated after wetting and mechanical action of the cloths. This also presupposes that the depressions in the fabric and the particle sizes of the wax component particles are co-ordinated with one another. The pits in the fabric should only be smaller or distinctly larger than the dispersed and subsequently applied wax component particles because simple dispersion of the particles during foaming would not otherwise be guaranteed.

The impregnated and dried cosmetic wipes produced are dry to the touch. The residual water content after drying or water content of the dry cosmetic wipes according to the invention is between 0.1 and 4% by weight, preferably between 0.5 and 3% by weight and more preferably between 0.8 and 2% by weight water, based on the weight of the dry non-impregnated carrier material.

Commercial Applications

The invention relates to preparations for impregnating cosmetic wipes which are characterized in that they contain
a) an emulsifier mixture containing nonionic and amphoteric surfactants in a quantity ratio of 10:1 to 1:1, based on the quantity of emulsifiers,
b) a mixture of wax components containing wax esters, partial glycerides and fatty alcohol ethoxylates and
c) at least one cationic polymer.
   The impregnating solutions generally contain
a) an emulsifier mixture containing nonionic and amphoteric surfactants in a quantity ratio of 5:1 to 1.5:1, based on the quantity of emulsifiers,
b) a mixture of wax components containing wax esters, partial glycerides and fatty alcohol ethoxylates in which the average particle size of the wax particles is at most 13 μm and
c) at least one cationic polymer.
   In a preferred embodiment, they contain
a) an emulsifier mixture containing nonionic and amphoteric surfactants in a quantity ratio of 4:1 to 2:1, based on the quantity of emulsifiers,
b) a mixture of wax components containing wax esters, partial glycerides and fatty alcohol ethoxylates in which the average particle size of the wax particles is at most 4 μm and
c) at least one cationic polymer,
d) polyols and
e) anionic surfactants.
   In a particularly preferred embodiment, they contain
a) an emulsifier mixture containing nonionic and amphoteric surfactants in a quantity ratio of 4:1 to 2:1, based on the quantity of emulsifiers,
b) a mixture of wax components containing wax esters, partial glycerides and fatty alcohol ethoxylates in which the average particle size of the wax particles is at most 2 μm,
c) at least one cationic polymer and

d) polyols.

TABLE 1a
Composition of the impregnating solutions -
quantities in % by weight actibe substance

| | | | Particularly |
|---|---|---|---|
| Component | Usual | Preferred | preferred |
| Wax ester | 0.05 to 10 | 0.5 to 8 | 1.5 to 6 |
| Fatty alcohol ethoxylates | 0.05 to 3.0 | 0.2 to 2 | 0.4 to 1.5 |
| Partial glycerides | 0.01 to 5 | 0.05 to 4 | 0.5 to 3 |
| Nonionic surfactants | 1 to 50 | 10 to 40 | 20 to 30 |
| Amphosurfactants, betaine | 0.5 to 25 | 2 to 15 | 4 to 10 |
| Polyols | 0 to 20 | 1 to 15 | 3 to 10 |
| Cationic polymers | 0.02 to 3 | 0.05 to 2.0 | 0.1 to 1.5 |

| | |
|---|---|
| Water | to 100 |

The preparations for the impregnating solutions may also be used as concentrates.

TABLE 1b
Concentrates for use in impregnating solutions -
quantities in % by weight active substance

| | | | | | Particularly |
|---|---|---|---|---|---|
| Commercial name | | INCI Name | Usual | Preferred | preferred |
| 1) | Cutina | Cetylpalmitate | 0.1 to 10 | 1 to 8 | 3 to 6 |
| 2) | Eumulgin B 10 | Beheneth-10 | 0.1 to 5 | 0.5 to 2 | 0.8 to 1.4 |
| 3) | Cutina HR | Hydrogenated castor oil | 0 to 5 | 0.1 to 2 | 0.3 to 0.8 |
| 4) | Cutina GMS | Glycerylpalmitate | 0.01 to 5 | 0.1 to 2 | 0.3 to 0.6 |
| 5) | Monomuls 90-O18 | Glyceryloleate | 0.1 to 5 | 1 to 3 | 1.6 to 2.2 |
| 6) | Plantacare 2000 UP | Decyl Glucoside | 1 to 50 | 5 to 40 | 20 to 25 |
| 7) | Dehyton K | Cocamidopropyl Betaine | 1 to 30 | 3 to 15 | 5 to 8 |
| 8) | Plantacare 818 UP | Coco-Glucoside | 0.1 to 20 | 1 to 10 | 1.8 to 6 |
| 9) | Citric acid | Citric acid | to pH 6 | to pH 6 | to pH 6 |
| 10) | Glycerol | Glycerine | 0 to 20 | 3 to 15 | 8 to 10 |
| 11) | Cosmedia Guar C 261 | Guar Hydroxypropyl | 0.1 to 3 | 0.5 to 2 | 0.8 to 1.4 |
| | | Trimonium Chloride | | | |

| | | |
|---|---|---|
| 12) | Preserved water | to 100.0 |

Production:

Components 1 to 4 and components 6 to 8 and 12 are heated to 80-85° C. or melted, mixed together and cold-stirred. Component 5 is added to the emulsion at 40 to 50° C. Components 10 and 11 are premixed and added to the emulsion with stirring after cooling to room temperature. 0.1% citric acid solution (9) is added to the preparation for pH adjustment from pH 5.2 to 6.8.

The concentrates may be used with water and other auxiliaries, the percentage content of the concentrates in the final impregnating solutions being from 1 to 99% by weight, preferably from 10 to 90% by weight and more preferably from 40 to 60% by weight, based on the impregnating solution.

The solutions and/or dispersions used for impregnation may additionally contain oil components, emulsifiers, softeners, refatting agents, polymers, silicone compounds, lecithins, phospholipids, biogenic agents, UV protection factors, antioxidants, deodorizers, antiperspirants, antidandruff agents, film formers, hydrotropes, solubilizers, preservatives, perfume oils, dyes and the like as further auxiliaries and additives.

Oil Components

Suitable oil components are, for example, Guerbet alcohols based on fatty alcohols containing 6 to 18 and preferably 8 to 10 carbon atoms, esters of linear C₆₋₂₂ fatty acids with linear or branched C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkyl hydroxycarboxylic acids with linear or branched C₆₋₂₂ fatty alcohols (cf. DE 19756377 A1), more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-/di-/triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, for example Dicaprylyl Carbonate (Cetiol® CC), Guerbet carbonates based on C₆₋₁₈ and preferably C₈₋₁₀ fatty alcohols, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, for example Dicaprylyl Ether (Cetiol® OE), ring opening products of epoxidized fatty acid esters with polyols, silicone oils (cyclomethicone, silicon methicone types, etc.) and/or aliphatic or naphthenic hydrocarbons, for example squalane, squalene or dialkyl cyclohexanes.

Other Emulsifiers

Suitable emulsifiers are, for example, other nonionic surfactants from at least one of the following groups:

products of the addition of 2 to 30 moles of ethylene oxide and/or 0 to 5 moles of propylene oxide onto linear C₈₋₂₂ fatty alcohols, C₁₂₋₂₂ fatty acids and alkyl phenols containing 8 to 15 carbon atoms in the alkyl group and alkylamines containing 8 to 22 carbon atoms in the alkyl group;

alkyl and/or alkenyl oligoglycosides containing 8 to 22 carbon atoms in the alk(en)yl group and ethoxylated analogs thereof;

products of the addition of 1 to 15 moles of ethylene oxide onto castor oil and/or hydrogenated castor oil;

products of the addition of 15 to 60 moles of ethylene oxide onto castor oil and/or hydrogenated castor oil;

partial esters of sorbitan with unsaturated, linear or saturated, branched fatty acids containing 12 to 22 carbon atoms and/or hydroxycarboxylic acids containing 3 to 18 carbon atoms and addition products thereof with 1 to 30 moles of ethylene oxide;

partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and addition products thereof with 1 to 30 moles of ethylene oxide;

mixed esters of pentaerythritol, fatty acids, citric acid and fatty alcohol according to DE 11 65 574 PS and/or mixed esters of fatty acids containing 6 to 22 carbon atoms, methyl glucose and polyols, preferably glycerol or polyglycerol,

mono-, di- and trialkyl phosphates and mono-, di- and/or tri-PEG-alkyl phosphates and salts thereof,

wool wax alcohols,

polysiloxane/polyalkyl/polyether copolymers and corresponding derivatives,

block copolymers, for example Polyethylene Glycol-30 Dipolyhydroxystearate;

polymer emulsifiers, for example Pemulen types (TR-1, TR-2) from Goodrich;

polyalkylene glycols and

glycerol carbonate.

Ethylene Oxide Addition Products

The addition products of ethylene oxide and/or propylene oxide onto fatty alcohols, fatty acids, alkylphenols or castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as refatting agents for cosmetic formulations from DE 2024051 PS.

Sorbitan Esters

Suitable sorbitan esters are sorbitan monoisostearate, sorbitan sesqui-isostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 moles of ethylene oxide onto the sorbitan esters mentioned are also suitable.

Polyglycerol Esters

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylol propane or pentaerythritol with lauric acid, cocofatty acid, tallow fatty acid, palmitic acid, stearic acid, oleic acid, behenic acid and the like optionally reacted with 1 to 30 moles of ethylene oxide.

Anionic Emulsifiers

Typical anionic emulsifiers are aliphatic C₁₂₋₂₂ fatty acids, such as palmitic acid, stearic acid or behenic acid for example, and C₁₂₋₂₂ dicarboxylic acids, such as azelaic acid or sebacic acid for example.

Amphoteric and Cationic Emulsifiers

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylamino-butyric acids, N-alkyliminodipropionic acids, N-hydroxyethyl-N-alkyl-amidopropyl glycines, N-alkyl taurines, N-alkyl sarcosines, 2-alkyl-aminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine. Finally, other suitable emulsifiers are cationic surfactants, those of the esterquat type, preferably methyl-quaternized difatty acid triethanolamine ester salts, being particularly preferred.

Polymers

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones. Other suitable polymers and thickeners can be found in Cosm. Toil., 108, 95 (1993).

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

UV Protection Factors and Antioxidants

UV protection factors in the context of the invention are, for example, organic substances (light filters) which are liquid or crystalline at room temperature and which are capable of absorbing ultraviolet or infrared radiation and of releasing the energy absorbed in the form of longer-wave radiation, for example heat. UV-B filters can be oil-soluble or water-soluble. The following are examples of oil-soluble substances:
3-benzylidene camphor or 3-benzylidene norcamphor and derivatives thereof, for example 3-(4-methylbenzylidene)-camphor as described in EP 0693471 B1;
4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);
esters of salicylic acid, preferably salicylic acid-2-ethylhexyl ester, salicylic acid-4-isopropylbenzyl ester, salicylic acid homomenthyl ester;
derivatives of benzophenone, preferably 2-hydroxy-4-methoxybenzo-phenone, 2-hydroxy-4-methoxy-4′-methylbenzophenone, 2,2′-dihydroxy-4-methoxybenzophenone;
esters of benzalmalonic acid, preferably 4-methoxybenzalmalonic acid di-2-ethylhexyl ester;
triazine derivatives such as, for example, 2,4,6-trianilino-(p-carbo-2′-ethyl-1′-hexyloxy)-1,3,5-triazine and Octyl Triazone as described in EP 0818450 A1or Dioctyl Butamido Triazone (Uvasorb® HEB);
propane-1,3-diones such as, for example, 1-(4-tert.butylphenyl)-3-(4′-methoxyphenyl)-propane-1,3-dione;
ketotricyclo(5.2.1.0)decane derivatives as described in EP 0694521 B1.

Suitable water-soluble substances are
2-phenylbenzimidazole-5-sulfonic acid and alkali metal, alkaline earth metal, ammonium, alkylammonium, alkanolammonium and glucammonium salts thereof;
sulfonic acid derivatives of benzophenones, preferably 2-hydroxy-4-methoxybenzophenone-5-sulfonic acid and salts thereof;
sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol® 1789) or 1-phenyl-3-(4′-isopropylphenyl)-propane-1,3-dione and the eneamine compounds described in DE 19712033 A1 (BASF). The UV-A and UV-B filters may of course also be used in the form of mixtures. Particularly favorable combinations consist of the derivatives of benzoylmethane, for example 4-tert.-butyl-4′-methoxydibenzoyl methane (Parsol® 1789) and 2-cyano-3,3-phenylcinnamic acid-2-ethylhexyl ester (Octocrylene) in combination with esters of cinnamic acid, preferably 4-methoxycinnamic acid-2-ethylhexyl ester and/or 4-methoxycinnamic acid propyl ester and/or 4-methoxycinnamic acid isoamyl ester. These combinations are advantageously combined with water-soluble filters such as, for example, 2-phenylbenzimidazole-5-sufonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolexe T2000 (Merck). So-called micro- or nanopigments are preferably used in sun protection products. Micronized zinc oxide is preferably used. Other suitable UV filters can be found in P. Finkel's review in SÖFW-Journal 122, 543 (1996) and in Parf. Kosm. 3, 11 (1999).

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Biogenic Agents

In the context of the invention, biogenic agents are, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, for example prune extract, bambara nut extract, and vitamin complexes.

Deodorants and Germ Inhibitors

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers.

Germ Inhibitors

Basically, suitable germ inhibitors are any substances which act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether(triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, farnesol, phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

Enzyme Inhibitors

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Odor Absorbers

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of ladanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic fragrances. Natural fragrances include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Antiperspirants

Antiperspirants reduce perspiration and thus counteract underarm wetness and body odor by influencing the activity of the eccrine sweat glands. Aqueous or water-free antiperspirant formulations typically contain the following ingredients:
astringent active principles,
oil components,
nonionic emulsifiers,
co-emulsifiers,
consistency factors,
auxiliaries in the form of, for example, thickeners or complexing agents and/or
non-aqueous solvents such as, for example, ethanol, -propylene glycol and/or glycerol.

Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine. Oil-soluble and water-soluble auxiliaries typically encountered in antiperspirants may also be present in relatively small amounts. Oil-soluble auxiliaries such as these include, for example,
inflammation-inhibiting, skin-protecting or pleasant-smelling essential oils,
synthetic skin-protecting agents and/or
oil-soluble perfume oils.

Typical water-soluble additives are, for example, preservatives, water-soluble perfumes, pH adjusters, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Film Formers

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Antidandruff Agents

Suitable antidandruff agents are Pirocton Olamin (1-hydroxy-4-methyl-6-(2,4,4-trimethylpentyl)-2-(1H)-pyridinone monoethanolamine salt), Baypival® (Climbazole), Ketoconazol® (4-acetyl-1-{4-[2-(2,4-dichlorophenyl)r-2-(1H-imidazol-1-ylmethyl)-1,3-dioxylan-c-4-ylmethoxyphenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

Swelling Agents

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

Insect Repellents

Suitable insect repellents are N,N-diethyl-m-toluamide, pentane-1,2-diol or Ethyl Butylacetylaminopropionate.

Hydrotropes

In addition, hydrotropes, for example ethanol, isopropyl alcohol or polyols, may be used to improve flow behavior during application. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 dalton;
technical oligoglycerol mixtures with a degree of self-condensation of 1.5 to 10 such as, for example, technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
aminosugars, for example glucamine;
dialcoholamines, such as diethaolamine or 2-aminopropane-1,3-diol.
   Preservatives

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”).

Perfume Oils and Aromas

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Suitable aromas are, for example, peppermint oil, spearmint oil, aniseed oil, Japanese anise oil, caraway oil, eucalyptus oil, fennel oil, citrus oil, wintergreen oil, clove oil, menthol and the like.

Dyes

Suitable dyes are any of the substances suitable and approved for cosmetic purposes as listed, for example, in the publication “Kosmetische Färbemittel” of the Farbstoffkommission der Deutschen Forschungs-gemeinschaft, Verlag Chemie, Weinheim, 1984, pages 81 to 106. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). Luminol may also be present as a luminescent dye. These dyes are normally used in concentrations of 0.001 to 0.1% by weight, based on the mixture as a whole.

### EXAMPLES

Various impregnating dispersions (Tables 3, 4) were prepared simply by mixing the components. Viscose/polyester cloths measuring 18.8 cm×14.8 cm as per specification (Table 2) were each moistened with 1-3.0 g of the dispersions and were then oven-dried for 2 h at max. 50° C.

TABLE 2
Specification for carrier material used for cosmetic wipes

| Test | Unit | Limit |
|---|---|---|
| Weight | g/m² | 80 ± 8.0 |
| Composition | % | 67% viscose |
| | | 33% polyester |
| Thickness | mm | 0.62 ± 0.06 |
| Tear strength MD* | N/50 mm | 130-160 |
| Tear strength TD** | N/50 mm | 17-21 |
| Elongation at break MD | % | 21 ± 8.0 |
| Elongation at break TD | % | 116 ± 25 |
| Tear strength, moist MD | N/50 mm | 90-120 |
| Tear strength, moist TD | N/50 mm | 15-19 |
| Absorption rate | secs. | 3.0 max |
| Absorption capacity | g/g | 5.5-7.5 |
| Wick effect MD | mm/2 mins | 100 ± 20 |
| pH | | 7.0 ± 1.0 |
| Drying residue | % | 8.0 max |
| Fluorescence | | corresponds |
| Water-soluble subst. | % | 0.5 max |
| Ether-soluble subst. | % | 0.5 max |
| Sulfate ash | % | 1.7 max |

*MD = machine direction - in the direction of travel in the production process
**TD = transverse direction - transversely of the direction of travel in the production process

Performance Test

Various impregnating solutions/dispersions (quantities in % by weight active substance, Table 3) were prepared simply by mixing the components. 67% viscose/33% polyester cloths (80 g/m²) measuring 18.8 cm×14.8 cm as per specification (Table 2) were each moistened with 1-3.0 g of the dispersions (Tables 3 and 4) and were then oven-dried for 2 h at max. 50° C.

In a panel test involving 4 people, the dry cosmetic wipes were prepared for use by moistening with an excess of water under a tap and rubbing between the hands for 20 s and were then used to clean the hands. An evaluation was made of the sensorial impression of the dry and moist wipe in the hands, the skin feel during use (moist), the skin feel after use and drying of the back of the hand (dry), the time needed for foaming, the structure of the foam and the foam volume. [Evaluation: 1=poor, 2=moderate, 3=good]

TABLE 3
Comparison of wipes with impregnating solution with/without wax dispersions

| Trade name | INCI name | C1 | 1 |
|---|---|---|---|
| Plantacare 2000 UP | Decyl Glucoside | 20 | 20 |
| Dehyton K | Cocamidopropyl Betaine | 9 | 9 |
| Lamesoft PO 65 | Coco Glucoside, Glyceryl Oleate | 3 | 3 |
| *microcrystalline, <0.5 | | | |
| Lamesoft PW 45 | Cetyl Palmitate, Beheneth-10, | 0 | 7 |
| *0.5-1.5 μm | Hydrogenated Castor Oil, Glyceryl | | |
| | Stearate | | |
| Glycerin | Glycerin | 4 | 4 |
| Cosmedia Guar C 261 | Guar Hydroxypropyl Trimonium Chloride | 0.5 | 0.5 |
| Water | to 100.0 | | |
| Sensorial impression | Poor = 1 | 2222 | 2222 |
| dry wipe | Moderate = 2 | | |
| | Good = 3 | | |
| Sensorial impression | Poor = 1 | 2211 | 3333 |
| moist wipe | Moderate = 2 | | |
| | Good = 3 | | |
| Skin feel during use (moist) | 1 = Slimy | 2121 | 3333 |
| | 2 = Creamily gentle | | |
| Skin feel of dry skin after | 1 = Sticky | 2112 | 2333 |
| use | 2 = Silkily soft | | |
| Time needed for foaming | >15 s = 1 | 2222 | 2233 |
| | <10 s = 3 | | |
| Foam structure | Loose, coarse-celled = 1 | 1111 | 3333 |
| | Firm, fine-celled = 3 | | |
| Foam volume | Low = 1 | 2222 | 2222 |
| | High = 3 | | |

TABLE 4a
Formulation Examples with different impregnating solutions,
quantities in % by weight active substance

| Commercial name | INCI Name | 1 | 2 | 3 | 4 | 5 | 6 | 7 |
|---|---|---|---|---|---|---|---|---|
| Plantacare 2000 UP | Decyl Glucoside | 23 | 20 | *20 | 20 | 20 | 20 | 20 |
| Dehyton K | Cocamidopropyl | 10 | 10 | 3 | 10 | 3 | 10 | 3 |
| | Betaine | | | | | | | |
| Lamesoft PO 65 | Coco-Glucoside, | 3 | 3 | 3 | 2 | 2 | 8 | 8 |
| *microcrystalline, <0.5 | Glyceryl Oleate | | | | | | | |
| Lamesoft PW 45 | Cetyl Palmitate, | 7 | 7 | 7 | 8 | 8 | 2 | 2 |
| *0.5-1.5 μm | Beheneth-10, | | | | | | | |
| | Hydrogenated Castor | | | | | | | |
| | Oil, Glyceryl Stearate | | | | | | | |
| Plantacare 818 UP | Coco-Glucoside | 2 | — | — | — | — | — | — |
| Citric acid | Citric acid | to pH | to pH | to pH | to pH | to pH | to pH | to pH |
| | | 6 | 6 | 6 | 6 | 6 | 6 | 6 |
| Glycerol | Glycerin | 5 | 3 | — | 3 | — | 3 | — |
| Cosmedia Guar C 261 | Guar Hydroxypropyl | 0.5 | 0.5 | 0.5 | 0.5 | 0.5 | 0.5 | 0.5 |
| | Trimonium Chloride | | | | | | | |

| | |
|---|---|
| Water | to 100 |

TABLE 4b
Formulation Examples with different impregnating solutions,
quantities in % by weight active substance

| Commercial name | INCI Name | 8 | 9 | 10 | 11 | 12 | 13 | 14 |
|---|---|---|---|---|---|---|---|---|
| Plantacare 2000 UP | Decyl Glucoside | 23 | 20 | 20 | 20 | 20 | 20 | 20 |
| Dehyton K | Cocamidopropyl | 10 | 10 | 3 | 10 | 3 | 10 | 3 |
| | Betaine | | | | | | | |
| Lamesoft PO 65 | Coco-Glucoside, | 5 | 5 | 5 | 1 | 10 | 12 | 1 |
| *microcrystalline, <0.5 | Glyceryl Oleate | | | | | | | |
| Lamesoft PW 45 | Cetyl Palmitate, | 5 | 5 | 5 | 10 | 1 | 1 | 12 |
| *0.5-1.5 μm | Beheneth-10, | | | | | | | |
| | Hydrogenated Castor | | | | | | | |
| | Oil, Glyceryl Stearate | | | | | | | |
| Plantacare 818 UP | Coco-Glucoside | 2 | — | — | — | — | — | — |
| Citric acid | Citric acid | to pH | to pH | to pH | to pH | to pH | to pH | to pH |
| | | 6 | 6 | 6 | 6 | 6 | 6 | 6 |
| Glycerol | Glycerin | 5 | 3 | — | 3 | — | 3 | — |
| Cosmedia Guar C 261 | Guar Hydroxypropyl | 0.5 | 0.5 | 0.5 | 0.5 | 0.5 | 0.5 | 0.5 |
| | Trimonium Chloride | | | | | | | |

| | |
|---|---|
| Water | to 100 |

1-11. (canceled)

12. A personal care wipe comprising a carrier substrate impregnated with a composition containing:
(a) an emulsifier containing a nonionic surfactant and an amphoteric surfactant in a ratio by weight of from about 10:1 to 1:1;
(b) a wax mixture containing a wax ester, a partial glyceride and a fatty alcohol ethoxylate;
(c) a cationic polymer;
(d) optionally, a polyol; and
(e) optionally, an anionic surfactant.

13. The wipe of claim 12 wherein the wax mixture has a mean particle size of at most 13 μm.

14. The wipe of claim 12 wherein the wax mixture has a mean particle size of at most 4 μm.

15. The wipe of claim 12 wherein the wax mixture has a mean particle size of at most 2 μm.

16. The wipe of claim 12 wherein the emulsifier is present in the composition in an amount of from about 15 to 55% by weight, based on the weight of the composition.

17. The wipe of claim 12 wherein the wax mixture is present in the composition in an amount of from about 0.2 to 35% by weight, based on the weight of the composition.

18. The wipe of claim 12 wherein the cationic polymer is present in the composition in an amount of from about 0.02 to 3% by weight, based on the weight of the composition.

19. The wipe of claim 12 wherein the polyol is present in the composition in an amount of from about 1 to 15% by weight, based on the weight of the composition.

20. The wipe of claim 12 wherein the wipe has a water content of from about 0.1 to 4% by weight.

21. The wipe of claim 12 wherein the carrier substrate is a woven or non-woven fabric made of natural or synthetic fibers.

22. A process for treating human skin comprising contacting the skin with a personal care wipe comprising a carrier substrate impregnated with a composition containing:
(f) an emulsifier containing a nonionic surfactant and an amphoteric surfactant in a ratio by weight of from about 10:1 to 1:1;
(g) a wax mixture containing a wax ester, a partial glyceride and a fatty alcohol ethoxylate;
(h) a cationic polymer;
(i) optionally, a polyol; and
(j) optionally, an anionic surfactant.

23. The process of claim 22 wherein the wax mixture has a mean particle size of at most 13 μm.

24. The process of claim 22 wherein the wax mixture has a mean particle size of at most 4 μm.

25. The process of claim 22 wherein the wax mixture has a mean particle size of at most 2 μm.

26. The process of claim 22 wherein the emulsifier is present in the composition in an amount of from about 15 to 55% by weight, based on the weight of the composition.

27. The process of claim 22 wherein the wax mixture is present in the composition in an amount of from about 0.2 to 35% by weight, based on the weight of the composition.

28. The process of claim 22 wherein the cationic polymer is present in the composition in an amount of from about 0.02 to 3% by weight, based on the weight of the composition.

29. The process of claim 22 wherein the polyol is present in the composition in an amount of from about 1 to 15% by weight, based on the weight of the composition.

30. The process of claim 22 wherein the wipe has a water content of from about 0.1 to 4% by weight.

31. A composition for use in treating human skin comprising:
(a) an emulsifier containing a nonionic surfactant and an amphoteric surfactant in a ratio by weight of from about 10:1 to 1:1;
(b) a wax mixture containing a wax ester, a partial glyceride and a fatty alcohol ethoxylate;
(c) a cationic polymer;
(d) optionally, a polyol; and
(e) optionally, an anionic surfactant.
